(19) United States
(12) Patent Application Publication
(10) Pub. No.: US 2023/0042011 A1
(43) Pub. Date: Feb. 9, 2023

(54) INTERATRIAL SHUNTS WITH ANCHORING MECHANISMS AND ASSOCIATED SYSTEMS AND METHODS

(71) Applicant: Shifamed Holdings, LLC, Campbell, CA (US)

(72) Inventors: Brian Fahey, Menlo Park, CA (US); William Jason Fox, San Mateo, CA (US); Stefan Schreck, Duvall, WA (US)

(21) Appl. No.: 17/792,686

(22) PCT Filed: Jan. 21, 2021
(86) PCT No.: PCT/US2021/014428

§ 371 Date: Jul. 13, 2022

Related U.S. Application Data
(60) Provisional application No. 62/963,683, filed on Jan. 21, 2020.

(51) Int. Cl. A61M 27/00

(52) U.S. Cl. CPC A61M 27/002; A61M 2210/125; A61M 2205/04

(57) ABSTRACT

The present technology relates to interatrial shunting systems and methods. In some embodiments, the present technology includes interatrial shunting systems that include a shunting element having a lumen extending therethrough that is configured to fluidly couple the left atrium and the right atrium when the shunting element is implanted in a patient. The system can also include an anchoring mechanism coupled to the shunting element and configured to secure the shunting element within the patients heart.

### CROSS-REFERENCE TO RELATED APPLICATION(S)

This application claims the benefit of U.S. Provisional Patent Application No. 62/963,683, filed Jan. 21, 2020, and incorporated herein by reference in its entirety.

### TECHNICAL FIELD

The present technology generally relates to implantable medical devices and, in particular, to implantable interatrial systems and associated methods for selectively controlling blood flow between the right atrium and the left atrium of a heart.

### BACKGROUND

Heart failure is a medical condition associated with the inability of the heart to effectively pump blood to the body. Heart failure affects millions of people worldwide, and may arise from multiple root causes, but is generally associated with myocardial stiffening, myocardial shape remodeling, and/or abnormal cardiovascular dynamics. Chronic heart failure is a progressive disease that worsens considerably over time. Initially, the body's autonomic nervous system adapts to heart failure by altering the sympathetic and parasympathetic balance. While these adaptations are helpful in the short-term, over a longer period of time they serve to make the disease worse.

Heart failure is a medical term that includes both heart failure with reduced ejection fraction (HFrEF) and heart failure with preserved ejection fraction (HFpEF). The prognosis with both HFpEF and HFrEF is poor; one-year mortality is 26% and 22%, respectively, according to one epidemiology study. In spite of the high prevalence of HFpEF, there remain limited options for HFpEF patients. Pharmacological therapies have been shown to impact mortality in HFrEF patients, but there are no similarly-effective evidence-based pharmacotherapies for treating HFpEF patients. Current practice is to manage and support patients while their health continues to decline.

A common symptom among heart failure patients is elevated left atrial pressure. In the past, clinicians have treated patients with elevated left atrial pressure by creating a shunt between the left and right atria using a blade or balloon septostomy. The shunt decompresses the left atrium (LA) by relieving pressure to the right atrium (RA) and systemic veins. Over time, however, the shunt typically will close or reduce in size. More recently, percutaneous interatrial shunt devices have been developed which have been shown to effectively reduce left atrial pressure. However, these percutaneous devices often have an annular passage with a fixed diameter which fails to account for a patient's changing physiology and condition. For this reason, existing percutaneous shunt devices may have a diminishing clinical effect after a period of time. Many existing percutaneous shunt devices typically are also only available in a single size that may work well for one patient but not another. Also, sometimes the amount of shunting created during the initial procedure is later determined to be less than optimal months later. Accordingly, there is a need for improved devices, systems, and methods for treating heart failure patients, particularly those with elevated left atrial pressure.

### BRIEF DESCRIPTION OF THE DRAWINGS

FIG. 1 is a schematic illustration of an interatrial device implanted in a heart and configured in accordance with an embodiment of the present technology.

FIG. 2 is a schematic illustration of an interatrial shunting system with flanges configured in accordance with an embodiment of the present technology.

FIG. 3 is a schematic illustration of an interatrial shunting system with a tether configured in accordance with another embodiment of the present technology.

FIG. 4 is a schematic illustration of an interatrial shunting system with an expandable structure configured in accordance with another embodiment of the present technology.

FIG. 5 is a schematic illustration of an interatrial shunting system with a plurality of anchoring elements configured in accordance with a further embodiment of the present technology.

FIG. 6 is a side cross-sectional view of a balloon-expandable interatrial shunting system configured in accordance with an embodiment of the present technology.

FIG. 7 is a plan view of an interatrial shunting system in an unrolled state and configured in accordance with an embodiment of the present technology.

FIGS. 8A-8D are side cross-sectional views of the system of FIG. 7 during various stages of implantation in a patient's heart in accordance with an embodiment of the present technology.

FIG. 8E is an end cross-sectional view of the system of FIG. 7 after implantation.

FIG. 9 is a side cross-sectional view of the system of FIG. 7 being adjusted to a greater diameter, in accordance with an embodiment of the present technology.

FIG. 10A is a perspective view of an adjustment device for adjusting the system of FIG. 7 to a smaller diameter in accordance with an embodiment of the present technology.

FIG. 10B is a side cross-sectional view of the system of FIG. 7 and the adjustment device of FIG. 10A during a first stage of operation.

FIG. 10C is an end view of the adjustment device of FIG. 10B.

FIG. 10D is a side cross-sectional view of the system of FIG. 7 and the adjustment device of FIG. 10A during a subsequent stage of operation.

FIG. 10E is an end view of the adjustment device of FIG. 10D.

### DETAILED DESCRIPTION

The present technology is generally directed to interatrial shunting systems. The systems can include a shunting element implantable into a patient at or adjacent a septal wall. The shunting element can fluidly connect a LA and a RA of the patient to facilitate blood flow therebetween. In some embodiments, the device further includes an anchoring mechanism coupled to the shunting element. The anchoring mechanism can be configured to secure the shunting element to a desired location in the patient's heart (e.g., to the septal wall between the LA and RA). The anchoring mechanism can include one or more flanges, tethers, anchoring elements (e.g., hooks, barbs), expandable or inflatable elements, or a combination thereof.

In some embodiments, the shunting element is balloon-expandable. For example, a method of implanting a shunting element in a patient can include inserting a balloon member into a lumen of the shunting element while the shunting element is in a contracted delivery configuration. The shunting element can be positioned within an aperture in the septal wall of the patient while in the delivery configuration. The shunting element can then be expanded to an expanded configuration by inflating the balloon member.

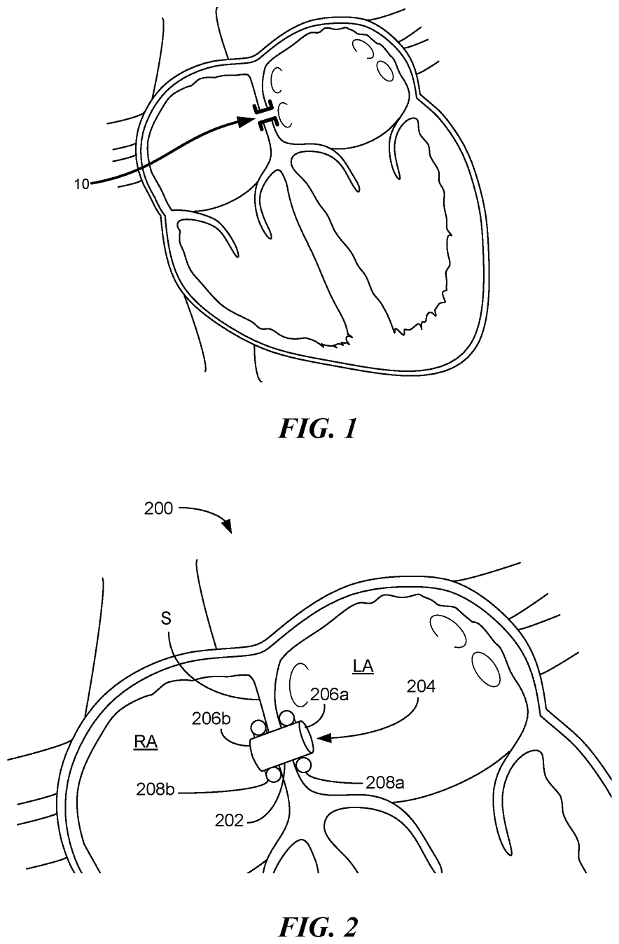

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-10E.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

As used herein, the terms “interatrial device,” “interatrial shunt device,” “IAD,” “IASD,” “interatrial shunt,” and “shunt” are used interchangeably to refer to a device that, in at least one configuration, includes a shunting element that provides a blood flow between a first region (e.g., a LA of a heart) and a second region (e.g., a RA or coronary sinus of the heart) of a patient. Although described in terms of a shunt between the atria, namely the left and right atria, one will appreciate that the technology may be applied equally to devices positioned between other chambers and passages of the heart, or between other parts of the cardiovascular system or other system. For example, any of the shunts described herein, including those referred to as “interatrial,” may be nevertheless used and/or modified to shunt between the LA and the coronary sinus, or between the right pulmonary vein and the superior vena cava. Moreover, while the disclosure herein primarily describes shunting blood from the LA to the RA, the present technology can be readily adapted to shunt blood from the RA to the LA to treat certain conditions, such as pulmonary hypertension. For example, mirror images of embodiments, or in some cases identical embodiments, used to shunt blood from the LA to the RA can be used to shunt blood from the RA to the LA in certain patients.

Although certain embodiments of the anchoring mechanisms described herein are discussed with respect to “preventing” movement of an interatrial shunting element relative to a portion of the patient's heart, one of skill in the art will appreciate that such anchoring mechanisms may still allow for movements that are expected to have little or no detrimental effect on the operation of the shunting element (e.g., movements that do not cause the shunting element to become dislodged from the septal wall).

The headings provided herein are for convenience only and do not interpret the scope or meaning of the claimed present technology.

### A. Interatrial Shunts for Treatment of Heart Failure

Heart failure can be classified into one of at least two categories based upon the ejection fraction a patient experiences: (1) HFpEF, historically referred to as diastolic heart failure or (2) HFrEF, historically referred to as systolic heart failure. One definition of HFrEF is a left ventricular ejection fraction lower than 35%-40%. Though related, the underlying pathophysiology and the treatment regimens for each heart failure classification may vary considerably. For example, while there are established pharmaceutical therapies that can help treat the symptoms of HFrEF, and at times slow or reverse the progression of the disease, there are limited available pharmaceutical therapies for HFpEF with only questionable efficacy.

In heart failure patients, abnormal function in the left ventricle (LV) leads to pressure build-up in the LA. This leads directly to higher pressures in the pulmonary venous system, which feeds the LA. Elevated pulmonary venous pressures push fluid out of capillaries and into the lungs. This fluid build-up leads to pulmonary congestion and many of the symptoms of heart failure, including shortness of breath and signs of exertion with even mild physical activity. Risk factors for HF include renal dysfunction, hypertension, hyperlipidemia, diabetes, smoking, obesity, old age, and obstructive sleep apnea. HF patients can have increased stiffness of the LV which causes a decrease in left ventricular relaxation during diastole resulting in increased pressure and inadequate filling of the ventricle. HF patients may also have an increased risk for atrial fibrillation and pulmonary hypertension, and typically have other comorbidities that can complicate treatment options.

Interatrial shunts have recently been proposed as a way to reduce elevated left atrial pressure, and this emerging class of cardiovascular therapeutic interventions has been demonstrated to have significant clinical promise. FIG. 2 shows the conventional placement of a shunt in the septal wall between the LA and RA. Most conventional interatrial shunts (e.g., shunt 10) involve creating a hole or inserting a structure with a lumen into the atrial septal wall, thereby creating a fluid communication pathway between the LA and the RA. As such, elevated left atrial pressure may be partially relieved by unloading the LA into the RA. In early clinical trials, this approach has been shown to improve symptoms of heart failure.

One challenge with many conventional interatrial shunts is determining the most appropriate size and shape of the shunt lumen. A lumen that is too small may not adequately unload the LA and relieve symptoms; a lumen that is too large may overload the RA and right-heart more generally, creating new problems for the patient. Moreover, the relationship between pressure reduction and clinical outcomes and the degree of pressure reduction required for optimized outcomes is still not fully understood, in part because the pathophysiology for HFpEF (and to a lesser extent, HFrEF) is not completely understood. As such, clinicians are forced to take a best guess at selecting the appropriately sized shunt (based on limited clinical evidence) and generally cannot adjust the sizing over time. Worse, clinicians must select the size of the shunt based on general factors (e.g., the size of the patient's anatomical structures, the patient's hemodynamic measurements taken at one snapshot in time, etc.) and/or the design of available devices rather than the individual patient's health and anticipated response. With many such traditional devices, the clinician does not have the ability to adjust or titrate the therapy once the device is implanted, for example, in response to changing patient conditions such as progression of disease. By contrast, interatrial shunting systems configured in accordance with embodiments of the present technology allow a clinician to select the size—perioperatively or post-implant—based on the patient.

B. Interatrial Shunting Systems with Anchoring Mechanism

As provided above, the present technology is generally directed to interatrial shunting systems. A system configured in accordance with an embodiment of the present technology can include, for example, a shunting element implantable into a patient at or adjacent a septal wall. The shunting element can fluidly connect a LA and a RA of the patient to facilitate blood flow therebetween. In some embodiments, the system further includes an anchoring mechanism for securing the shunting element to the septal wall and preventing the shunting element from becoming dislodged.

FIG. 2, for example, is a schematic illustration of an interatrial shunting system 200 (“system 200”) with flanges configured in accordance with an embodiment of the present technology. The system 200 includes a shunting element 202 defining a lumen 204 therethrough. The shunting element 202 can include a first end portion 206a positioned in the left atrium LA and a second end portion 206b positioned in the right atrium RA. Accordingly, when implanted in the septal wall S, the system 200 fluidly connects the left atrium LA and the right atrium RA via the lumen 204. When the system 200 is implanted, blood can flow through the lumen 204 from the left atrium LA to the right atrium RA.

The system 200 further includes a first flange 208a and a second flange 208b coupled to the shunting element 202. The first and second flanges 208a-b can collectively serve as an anchoring mechanism for securing the shunting element 202 within the septal wall S. In some embodiments, the first and second flanges 208a-b are both annular-shaped structures extending partially or completely around the circumference of the external surface of the shunting element 202. The first flange 208a, for example, can be coupled to or near the first end portion 206a of shunting element 202 and the second flange 208b can be coupled to or near the second end portion 206b. As a result, when the shunting element 202 is implanted in the septal wall S, the first flange 208a can be positioned against the left atrial side of the septal wall S and the second flange 208b can positioned against the right atrial side of the septal wall S. The first and second flanges 208a-b are configured to engage and press against the septal wall S to prevent the shunting element 202 from becoming dislodged.

In some embodiments, the first and second flanges 208a-b are inflatable structures configured to be filled with a fluid (e.g., a gas or liquid). Prior to implantation of the shunting element 202, the first and second flanges 208a-b can be in a partially or completely deflated state, e.g., to facilitate delivery into the patient's heart. Once the shunting element 202 is positioned in the septal wall S, the first and second flanges 208a-b can be partially or completely filled with a fluid to expand them into an inflated state (e.g., as shown in FIG. 2). The inflated first and second flanges 208a-b can engage and press against the septal wall S to secure the shunting element 202 therein.

Various methods can be used to inflate the first and second flanges 208a-b with fluid after implantation in the patient's heart. For example, the first and second flanges 208a-b can be temporarily attached to a fill line (not shown) or other like structure for introducing fluid. The fill line can be removed once the first and second flanges 208a-b have been inflated to the desired volume with a fluid. Optionally, the fluid can be a curable material such that the fluid within the first and second flanges 208a-b can be cured (e.g., by application of light, heat, a cross-linking agent, etc.) to increase mechanical strength and/or reduce the likelihood of fluid leakage.

In some embodiments, at least one of the first and second flanges 208a-b can be made of and/or include an expandable material. The expandable material can initially be in a compressed and/or low-profile configuration that is relatively small in size to facilitate delivery into the patient's heart. After one or more delivery steps (e.g., unsheathing of at least a portion of the system 200 from a delivery catheter or other tool), the expandable material of the first and/or second flange 208a-b can transform into an expanded configuration that is relatively large in size to anchor the shunting element 202 within the patient's heart. For example, the expandable material can be a sponge-like material that is in a low-profile configuration when dried and/or compressed. The sponge-like material can be exposed to a fluidic environment during and/or after delivery into the heart (e.g., when exposed to blood from the patient's body, injected with a fluid and/or other expanding material, etc.), thus causing the sponge-like material to absorb fluid and increase in size to the expanded configuration.

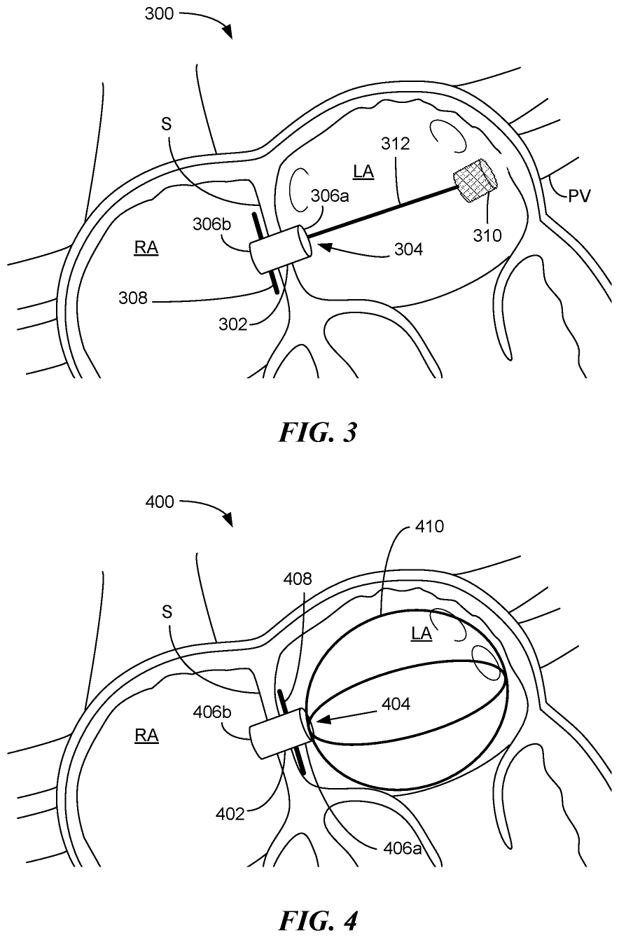

FIG. 3 is a schematic illustration of an interatrial shunting system 300 including a tether 312 and configured in accordance with another embodiment of the present technology. The components of the system 300 can be generally similar to the components of the system 200 described with respect to FIG. 2. For example, the system 300 includes a shunting element 302 defining a lumen 304 therethrough, and having a first end portion 306a positioned in the left atrium LA and a second end portion 306b positioned in the right atrium RA.

The system can further include a flange 308 coupled to the shunting element 302, an anchoring element 310 coupled to a portion of the heart, and a tether 312 connecting the shunting element 302 to the anchoring element 310. The flange 308, anchoring element 310, and tether 312 can collectively serve as an anchoring mechanism for securing the shunting element 302 to the septal wall S. The flange 308, for example, can be an annular-shaped structure configured to engage the septal wall S to prevent displacement of the shunting element 302. The flange 308 can extend partially or completely around the circumference of the external surface of the shunting element 302. The anchoring element 310 can be a stent, basket, cage, hook, barb, or any other structure that can be fastened to a portion of the patient's heart (e.g., to an internal surface of a heart chamber). The tether 312 can be any elongate structure suitable for connecting the shunting element 302 to the anchoring element 310. In some embodiments, the tether 312 is made of a material having sufficient strength and/or elasticity (e.g., a polymer, a metal, a composite, etc.) to accommodate stresses from the contractile motions of the heart chamber, e.g., without fracturing and/or plastically deforming.

In the illustrated embodiment, the flange 308 is coupled to or near the second end portion 306b of the shunting element 302, and is positioned against the right atrial side of the septal wall S. The anchoring element 310 can be a stent that is secured to an inner surface of the left atrium LA, e.g., near or within a pulmonary vein PV. The tether 312 can be coupled to anchoring element 310 and the first end portion 306a of the shunting element 302. The tension in the tether 312 can prevent the shunting element 302 from moving into the right atrium RA, while the flange 308 can prevent the shunting element 302 from being pulled into the left atrium LA. As a result, the shunting element 302 can be anchored within the septal wall S.

FIG. 4 is a schematic illustration of an interatrial shunting system 400 with an expandable structure 410 configured in accordance with another embodiment of the present technology. The components of the system 400 can be generally similar to the components of the other interatrial shunting systems described herein. For example, the system 400 includes a shunting element 402 defining a lumen 404 therethrough, and having a first end portion 406a positioned in the left atrium LA and a second end portion 406b positioned in the right atrium RA.

To secure the shunting element 402 to the septal wall S, the system 400 further includes an anchoring mechanism including a flange 408 and an expandable structure 410. The flange 408 can extend partially or completely around the circumference of the external surface of the shunting element 402. In some embodiments, the flange 408 is positioned against and/or engages the septal wall S to prevent displacement of the shunting element 402. The expandable structure 410 can be coupled to an end portion of the shunting element 402, such that the flange 408 is positioned between the expandable basket structure 410 and the septal wall S.

The expandable structure 410 can be a cage, basket, mesh, balloon, stent, or any other structure that can be transformed between a low-profile configuration and an expanded configuration (e.g., as shown in FIG. 4). In some embodiments, prior to implantation, the expandable structure 410 can be in the low-profile/contracted configuration, e.g., to facilitate delivery into the patient's heart. After the shunting element 402 has been positioned, the expandable structure 410 can be deployed into the expanded configuration. When in the expanded configuration, the expandable structure 410 can conform to and/or engage the inner surface of a heart chamber (e.g., the left atrium LA). The expandable structure 410 can apply a force to the shunting element 402 to prevent it from becoming dislodged from the septal wall S. In some embodiments, the expandable structure 410 is made of a material having sufficient strength and/or elasticity (e.g., a polymer, a metal, a composite, etc.) to accommodate stresses from the contractile motions of the heart chamber, e.g., without fracturing and/or plastically deforming. The expandable structure 410 can be configured such that, when it is in the expanded configuration, it does not substantially impact (e.g., adversely affect) flow of blood through the left atrium LA (e.g., if the expandable structure 410 is a balloon, it can have a toroidal shape, etc.).

In the illustrated embodiment, the expandable structure 410 is configured as a cage coupled to the first end portion 406a of the shunting element 402, and is positioned within the left atrium LA. When expanded, the expandable structure 410 can apply an outwardly-directed force against the walls of the left atrium LA. This force can also be applied to the shunting element 402 to prevent the shunting element 402 from moving into the left atrium LA. The flange 408 can be positioned against the left atrial side of the septal wall S to prevent the shunting element 402 from being pushed into the right atrium RA. As a result, the shunting element 402 can be anchored within the septal wall S. Optionally, in other embodiments the flange 408 can be omitted such that the shunting element 402 is secured in place only by the expandable structure 410.

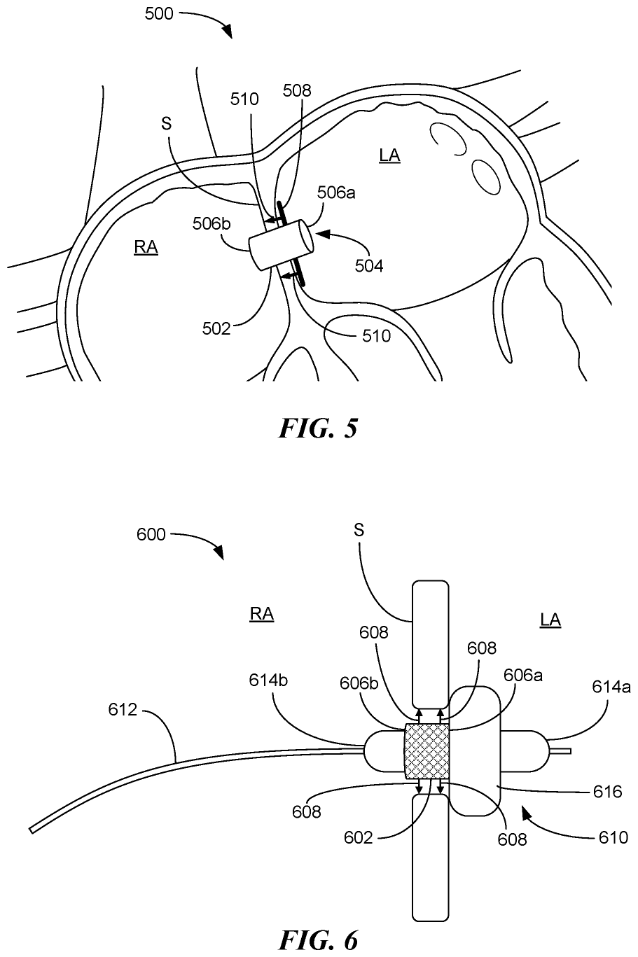

FIG. 5 is a schematic illustration of an interatrial shunting system 500 with a plurality of anchoring elements 510 configured in accordance with a further embodiment of the present technology. The components of the system 500 can be generally similar to the components of the other interatrial systems described herein. For example, the system 500 includes a shunting element 502 defining a lumen 504 therethrough, and having a first end portion 506a positioned in the left atrium LA and a second end portion 506b positioned in the right atrium RA.

To secure the shunting element 502 to the septal wall S, the system 500 further includes an anchoring mechanism including a flange 508 and a plurality of anchoring elements 510. The flange 508 can extend partially or completely around the circumference of the external surface of the shunting element 502. In some embodiments, the flange 508 is positioned against and/or engages the septal wall S to prevent displacement of the shunting element 502. For example, in the illustrated embodiment, the flange 508 is positioned within the left atrium LA and engages the left atrial side of the septal wall S, thereby preventing the shunting element 502 from moving into the right atrium RA.

The anchoring elements 510 can include hooks, barbs, or any other structure suitable for penetrating into a portion of the heart (e.g., the septal wall S). In the illustrated embodiment, the anchoring elements 510 are positioned on the flange 508 and are embedded into the tissue of the left atrial side of the septal wall S, thereby preventing the shunting element 502 from moving into the left atrium LA. Although FIG. 5 illustrates two anchoring elements 510, in other embodiments, the system 500 can include a different number of anchoring elements 510 (e.g., one, three, four, five, or more).

In some embodiments, the flange 508 is an expandable (e.g., inflatable) structure. Prior to implantation, the flange 508 can initially be in a low-profile/contracted configuration, e.g., to facilitate delivery into the patient's heart. The anchoring elements 510 can likewise be folded or otherwise contracted, e.g., to prevent tissue injury during the delivery process. After implantation, the flange 508 can be deployed to an expanded configuration (e.g., as shown in FIG. 5) to anchor the shunting element 502 in the septal wall S. The anchoring elements 510 can be deployed with the expansion of the flange 508 to embed into the septal wall S.

The anchoring mechanisms described above with respect to FIGS. 2-5 can be used to anchor an adjustable interatrial shunting system. For example, any of the shunting elements and/or lumens described above may be adjustable to control the flow of fluid through the shunt. Despite these adjustments, the anchoring mechanisms prevent substantial unwanted movement of the shunting elements and/or lumens (e.g., ejection from the septal wall), while still permitting the shunting elements, lumens, or other actuation mechanisms (not shown) to move to adjust the flow resistance through the system. Examples of adjustable interatrial shunting systems that can be used with the anchoring mechanisms described herein are described in International Patent Application Nos. PCT/US2020/038549, PCT/US2020/049996, PCT/US/2020/063360, and PCT/US2020/064529, the disclosures of which are incorporated by reference herein in their entireties.

In some embodiments, the interatrial shunting systems described herein are balloon-expandable. For example, a shunting element of an interatrial shunting system can have a first diameter when in a contracted configuration, and a second, greater diameter when in an expanded configuration. The shunting element can be introduced into the patient's heart while in the contracted configuration, and subsequently deployed to the expanded configuration using a balloon member.

FIG. 6 is a side cross-sectional view of a balloon-expandable interatrial shunting system 600 configured in accordance with an embodiment of the present technology. The components of the system 600 can be generally similar to the components of the other interatrial systems described herein. For example, the system 600 includes a shunting element 602 defining a lumen therethrough, and having a first end portion 606a positioned in the left atrium LA and a second end portion 606b positioned in the right atrium RA.

The system 600 can further include a plurality of anchoring elements 608 for securing the shunting element 602 in place. The anchoring elements 608 can include hooks, barbs, or any other structure suitable for penetrating into a portion of the heart (e.g., the septal wall S). In the illustrated embodiment, the anchoring elements 608 are coupled to the external surface of the shunting element 602 between the first and second end portions 606a-b. The anchoring elements 608 can extend radially outward from the shunting element 602 to embed into the tissue of the septal wall S surrounding the shunting element 602. Although FIG. 6 illustrates four anchoring element 608, in other embodiments, the system 600 can include a different number of anchoring elements 608 (e.g., one, two, three, five, or more).

In some embodiments, the system 600 is configured to be expandable such that the shunting element 602 and/or anchoring elements 608 can be transformed from a low-profile/contracted configuration (e.g., for delivery) to an expanded configuration (e.g., after implantation). For example, the expansion can be mechanically actuated by a delivery tool (e.g., a balloon) and/or a user (e.g., a clinician). As another example, one or more components of the system 600 (e.g., the shunting element 602 and/or anchoring elements 608) can be made of a shape memory material that expands upon application of energy (e.g., heat). In some embodiments, the system 600 is self-expanding. For example, one or more components of the system 600 (e.g., the shunting element 602 and/or anchoring elements 608) can be made of an elastic or superelastic material (e.g., nitinol) such that the component(s) are initially in a compressed and/or low-profile configuration for delivery and transformable into an expanded or deployed configuration at a target implantation site within the patient.

In some embodiments, the system 600 is expandable via a balloon. For example, as shown in in FIG. 6, the system 600 can be deployed using a balloon member 610 mounted on a delivery catheter 612. The balloon member 610 can include a first narrowed segment 614a, a second narrowed segment 614b, and a widened segment 616 disposed between the first and second narrowed segments 614a-b. Prior to implantation, the shunting element 602 can be positioned around a portion of the balloon member 610 (e.g., around the second narrowed segment 614b). The balloon member 610 can be partially or completely deflated, such that the shunting element 602 is initially in a contracted configuration. In some embodiments, the anchoring elements 608 are also initially in a retracted and/or low-profile configuration, e.g., to facilitate delivery, repositioning, and/or placement of the system 600 while avoiding inadvertent tissue damage.

The shunting element 602 can then be introduced into the patient's heart, e.g., into an aperture formed in the septal wall S between the left atrium LA and the right atrium RA. The balloon member 610 can then be inflated to expand the shunting element 602 into the expanded configuration (e.g., as shown in FIG. 6). In some embodiments, the widened segment 616 is used to align the shunting element 602 with the septal wall S. For example, the balloon member 610 can be shaped such that when the widened segment 616 is positioned against the left atrial side of the septal wall S, the second narrowed segment 614b carrying the shunting element 602 is positioned within the aperture formed in the septal wall S.

The anchoring elements 608 can be transformed between the retracted and/or low-profile configuration and an expanded and/or operating configuration to anchor the shunting element 602 within the septal wall S. The transformation of the anchoring elements 608 can occur automatically when the shunting element 602 is expanded. In other embodiments, the transformation can be actuated by a user (e.g., by rotation of a dial, pressing of a button, expansion via inflation of the balloon member 610, etc.). Once the shunting element 602 is secured, the balloon member 610 can be deflated and withdrawn from the patient's heart.

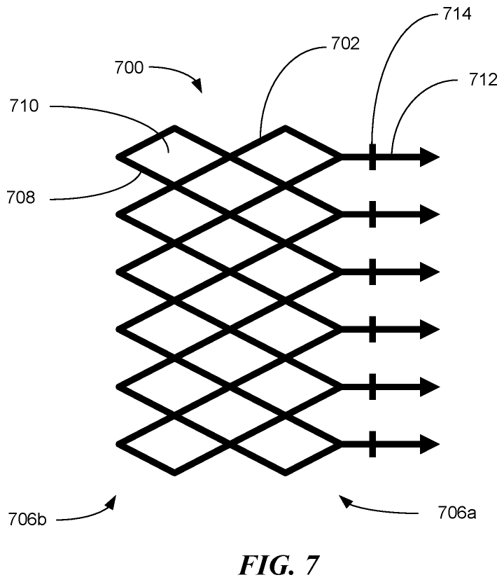

FIG. 7 is a plan view of an interatrial shunting system 700 in an unrolled state and configured in accordance with an embodiment of the present technology. The components of the system 700 can be generally similar to the components of the other interatrial systems described herein. For example, the system 700 includes a shunting element 702 having a first end portion 706a and a second end portion 706b. In the illustrated embodiment, the shunting element 702 is configured as a stent (e.g., a cobalt chromium stent) formed from a plurality of struts 708. The struts 708 can be interconnected with each other so as to form a plurality of closed cells 710. Although FIG. 7 shows the closed cells 710 as being diamond-shaped, in other embodiments, other cell geometries can be used (e.g., square, rectangular, rectilinear, polygonal, curvilinear, etc.).

The system 700 further includes a plurality of anchoring elements 712 (e.g., hooks, barbs, etc.) for securing the shunting element 702 to a portion of the heart (e.g., to the septal wall). Although FIG. 7 illustrates six anchoring elements 712, in other embodiments, the system 700 can include a different number of anchoring elements 712 (e.g., one, two, three, four, five, or more). In the illustrated embodiment, the anchoring elements 712 are coupled to the first end portion 706a of the shunting element 702. The anchoring elements 712 can be oriented with their longitudinal axes aligned with a longitudinal axis of the shunting element 702. In some embodiments, each anchoring element 712 includes an alignment feature 714 (e.g., a stop or flange) for limiting the penetration depth of the anchoring element 712 into tissue.

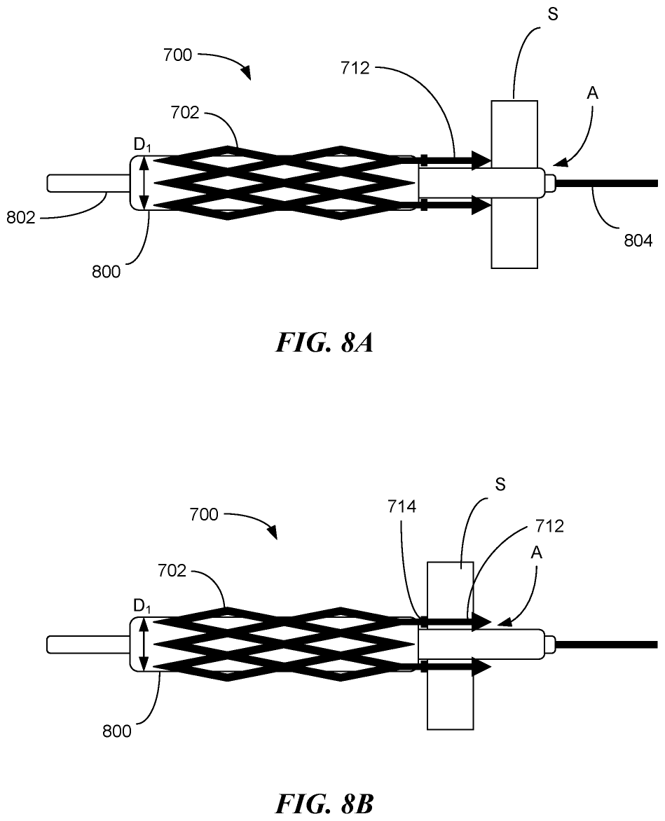

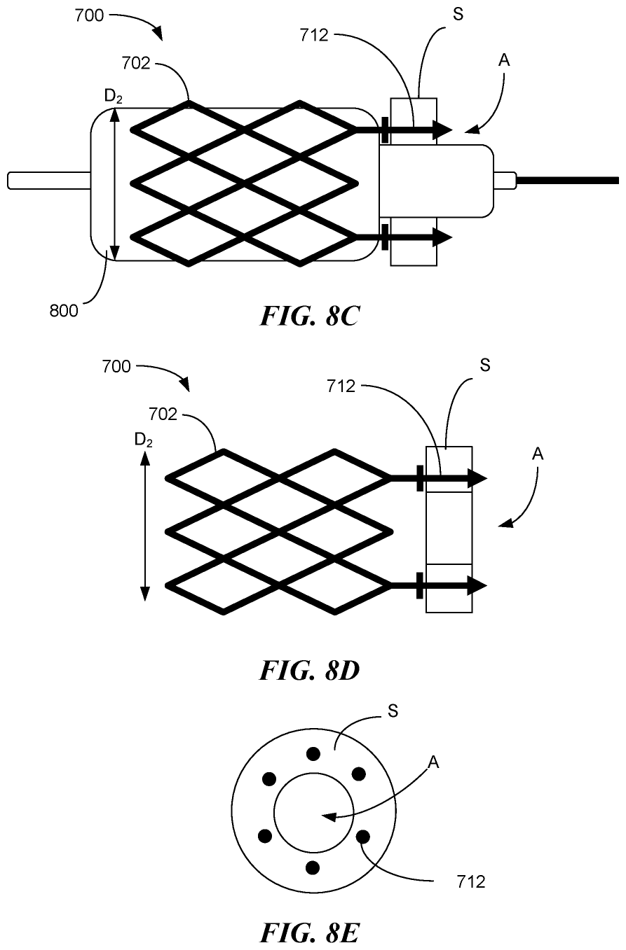

FIGS. 8A-8E illustrate steps of a method for implanting the interatrial shunting system 700 described with respect to FIG. 7 in a patient's heart in accordance with an embodiment of the present technology. More specifically, FIGS. 8A-8D are side cross-sectional views of the system 700 during various stages of the implantation process, while FIG. 8E is an end cross-sectional view of the system 700 after implantation.

Referring first to FIG. 8A, the shunting element 702 can initially be in a low-profile delivery configuration having a first diameter D₁. The shunting element 702 can be positioned around a balloon or expandable member 800, which can likewise initially be in a partially or completely deflated state. The balloon member 800 can be coupled to a delivery catheter 802. The shunting element 702 and balloon member 800 can be introduced into the patient's heart (e.g., using a guidewire 804) and positioned such that the anchoring elements 712 of the shunting element 702 extend towards the septal wall S. In some embodiments, a portion of the balloon member 800 and/or delivery catheter 802 are introduced into an aperture A in the septal wall S to center the shunting element 702 relative to the aperture A. Optionally, a portion of the shunting element 702 or the entire shunting element 702 can be introduced into the aperture A.

Referring to FIG. 8B, in a subsequent step the shunting element 702 can be advanced toward the septal wall S such that anchoring elements 712 penetrate into the portion of the septal wall S surrounding the aperture A. The penetration depth of the anchoring elements 712 can be limited by the alignment features 714. Although FIG. 8B shows the anchoring elements 712 penetrating completely through the septal wall S and extending partially out through the outer side, in other embodiments different configurations of the anchoring elements 712 can be used. For example, the anchoring elements 712 can penetrate only partially through the septal wall S. As another example, the anchoring elements 712 can be introduced through the aperture A and can engage the distal side (e.g., the left atrial side) of the septal wall S. In yet another example, the anchoring elements 712 can engage the septal wall S without penetrating into the tissue (e.g., via magnets, clips, etc.).

Referring next to FIG. 8C, the balloon member 800 can be inflated, thus expanding the shunting element 702 to an expanded configuration having a second diameter D₂ greater than the first diameter D₁. The expansion of the balloon member 800 can also expand the diameter of the aperture A in the septal wall S. The anchoring elements 712 can be expanded radially outward along with the shunting element 702. In some embodiments, the anchoring element 712 can maintain the shunting element 702 and/or aperture A in the expanded configuration.

Referring to FIGS. 8D and 8E together, in a subsequent step the balloon member 800, delivery catheter 802, and guidewire 804 can be withdrawn from the patient's heart, leaving the shunting element 702 secured to the septal wall S via anchoring elements 712. The anchoring elements 712 can also maintain the diameter of the shunting element 702 after the balloon member 800 has been removed. As shown in FIG. 8E, the anchoring elements 712 can be radially distributed around the aperture A. In some embodiments, the native tissue of the septal wall S surrounding the aperture A is preserved, which is expected to reduce pannus formation and/or occlusion of the shunting element 702. In other embodiments, the shunting element 702 can be positioned within the aperture A and engage directly with the tissue of the septal wall S. Optionally, the shunting element 702 can include an outer and/or inner sleeve, sheath, jacket, etc. (not shown) configured to provide an enclosed lumen that, when expanded, has a size identical or similar to the size of the aperture A. In such embodiments, the lumen and aperture A can form a continuous or generally continuous fluidic channel between the LA and the RA.

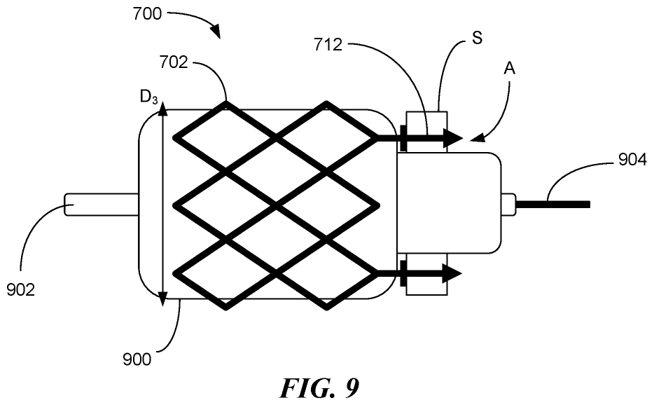

FIG. 9 is a side cross-sectional view of the system 700 being adjusted to a greater diameter, in accordance with an embodiment of the present technology. Adjustments to the diameter of the shunting element 702 can be made after implantation (e.g., in response to shunt narrowing and/or occlusion, to adjust to changing conditions, in accordance with a treatment plan, etc.). For example, the diameter of the shunting element 702 can be increased to a third, greater diameter D₃ to permit increased blood flow through the shunting element 702. To expand the shunting element 702, a balloon member 900 can be introduced into the patient's heart and positioned within the shunting element 702 (e.g., using a delivery catheter 902 and/or guidewire 904). The balloon member 900, delivery catheter 902, and/or guidewire 904 can be the same components used to implant the shunting element 702, or can be different components. The size of the balloon member 900 can be selected based on the particular desired diameter for the shunting element 702.

The balloon member 900 can initially be in a partially or completely deflated state to facilitate introduction into the patient's heart and insertion into the shunting element 702. Once properly positioned, the balloon member 900 can be inflated to expand the shunting element 702 to the third diameter D₃, as shown in FIG. 9. The anchoring elements 712 can be expanded radially outwards along with the shunting element 702. Subsequently, the balloon member 900, delivery catheter, and guidewire 904 can be withdrawn, leaving the anchoring elements 712 to secure the shunting element 702 in the newly expanded configuration.

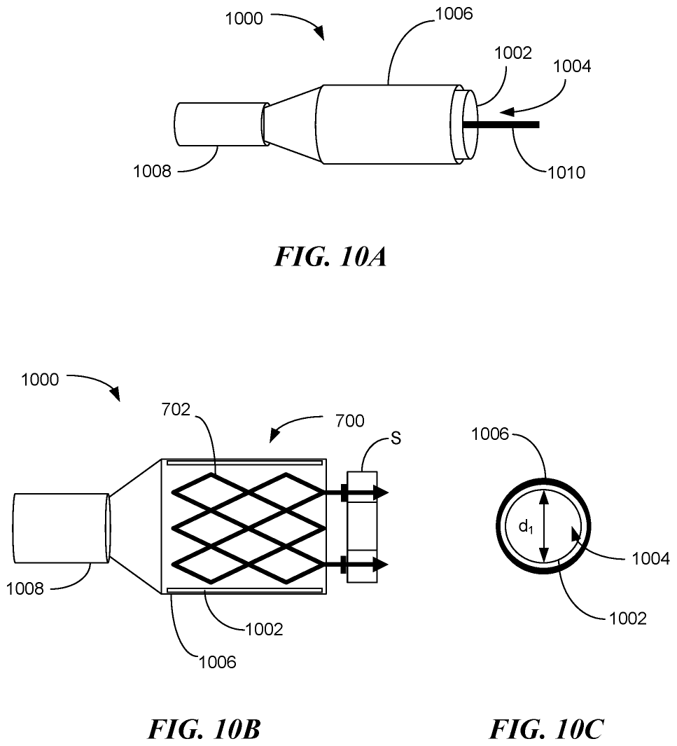

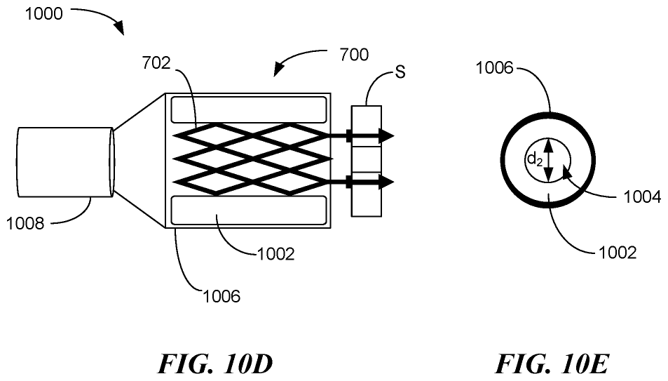

FIGS. 10A-10E illustrate adjustment of the system 700 to a smaller diameter using an adjustment device 1000 configured in accordance with an embodiment of the present technology. FIG. 10A, for example, is a perspective view of the adjustment device 1000. FIGS. 10B and 10C are a side cross-sectional view and end view, respectively, of the system 700 and adjustment device 1000 during a first stage of operation. FIGS. 10D and 10E are a side cross-sectional view and end view, respectively, of the system 700 and adjustment device 1000 during a subsequent stage of operation.

Referring first to FIG. 10A, the adjustment device 1000 includes a balloon member 1002 having a tubular shape with a lumen 1004 extending therethrough. The balloon member 1002 can be positioned within a sleeve 1006. The sleeve 1006 can be a mesh, stent, or any other structure capable of self-expanding to a predetermined diameter. The balloon member 1002 and sleeve 1006 can both be collapsed into a low-profile configuration for positioning within a delivery catheter 1008. The delivery catheter 1008 can be advanced over a guidewire 1010 in order to introduce the balloon member 1002 and sleeve 1006 into the patient's heart.

Referring to FIGS. 10B and 10C together, once the delivery catheter 1008 is positioned adjacent to the shunting element 702, the balloon member 1002 and sleeve 1006 can be advanced distally out of the delivery catheter 1008 and around the shunting element 702. As the sleeve 1006 exits the delivery catheter 1008, it can self-expand into an expanded configuration with the predetermined diameter. The balloon member 1002 can remain in a deflated state. In some embodiments, the balloon member 1002 is coupled to the interior surface of the sleeve 1006. As a result, the expansion of the sleeve 1006 also pulls the balloon 1002 radially outward so that the lumen 1004 has a first lumen diameter di that is sufficiently large to accommodate the shunting element 702.

Referring to FIGS. 10D and 10E together, in a subsequent stage of operation the balloon member 1002 can be inflated while positioned around the shunting element 702. The balloon member 1002 can expand radially inward as it inflates, thereby decreasing the size of the lumen 1004 to a second, smaller lumen diameter dz. In some embodiments, the sleeve 1006 restricts the outward radial expansion of the balloon member 1002, such that the balloon member 1002 expands inward rather than outward as it inflates. The inward expansion of the balloon member 1002 can engage with and compress the shunting element 702 to the smaller diameter dz. Subsequently, the balloon member 1002 and sleeve 1006 can be withdrawn from the shunting element 702, collapsed back into their contracted configurations, and retracted into the delivery catheter 1008.

As one of skill in the art will appreciate from the disclosure herein, various components of the interatrial shunting systems described above can be omitted without deviating from the scope of the present technology. Likewise, additional components not explicitly described above may be added to the interatrial shunting systems without deviating from the scope of the present technology. Accordingly, the systems described herein are not limited to those configurations expressly identified, but rather encompasses variations and alterations of the described systems.

### Examples

Several aspects of the present technology are set forth in the following examples:

1. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism coupled to the shunting element, wherein the anchoring mechanism is configured to secure the shunting element to a septal wall between the left atrium and the right atrium, and wherein the anchoring mechanism comprises one or more anchoring elements configured to penetrate into the septal wall.

2. The system of example 1 wherein the one or more anchoring elements comprise one or more hooks or barbs.

3. The system of example 1 or 2 wherein the anchoring mechanism further comprises a flange coupled to the shunting element, and the one or more anchoring elements are positioned on the flange.

4. The system of any of examples 1-3 wherein the one or more anchoring elements are positioned on an external surface of the shunting element.

5. The system of any of examples 1-3 wherein the one or more anchoring elements are positioned on an end portion of the shunting element.

6. The system of any of examples 1-5 wherein the shunting element comprises a stent having a plurality of struts.

7. The system of any of examples 1-6 wherein the anchoring mechanism further comprises one or more alignment features configured to limit a penetration depth of the one or more anchoring elements into the septal wall.

8. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism coupled to the shunting element, wherein the anchoring mechanism is configured to secure the shunting element to a septal wall between the left atrium and the right atrium, and wherein the anchoring mechanism comprises one or more inflatable flanges extending circumferentially around the shunting element.

9. The system of example 8 wherein the one or more inflatable flanges comprise a first inflatable flange configured to be positioned against a left atrial side of the septal wall and a second inflatable flange configured to be positioned against a right atrial side of the septal wall.

10. The system of example 8 or 9 wherein the one or more inflatable flanges are configured to receive a curable inflation medium.

12. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism configured to secure the shunting element to a septal wall between the left atrium and the right atrium, the anchoring mechanism including:
   a flange coupled to the shunting element and configured to be positioned against a right atrial side of the septal wall,
   an anchoring element configured to be at least partially spaced apart from the septal wall, and
   a tether connecting the shunting element to the anchoring element.

12. The system of example 12 wherein the anchoring element comprises a stent positioned near or within a pulmonary vein.

13. The system of example 11 or 12 wherein the anchoring element comprises an expandable structure.

14. The system of example 13 wherein the expandable structure comprises a cage or a basket.

15. A method of implanting a shunting element in a septal wall between a left atrium and a right atrium in a patient, the method comprising:
positioning the shunting element around a balloon member while the shunting element is in a contracted configuration having a first diameter;
introducing the shunting element into or near an aperture in the septal wall;
expanding the shunting element to an expanded configuration having a second diameter by inflating the balloon member;
securing the shunting element to the septal wall using one or more anchoring elements; and
after securing the shunting element to the septal wall, adjusting the shunting element to a third diameter that is different than the second diameter.

16. The method of example 15 wherein adjusting the shunting element comprises expanding the shunting element to the third diameter, wherein the third diameter is greater than the second diameter.

17. The method of example 16 wherein the shunting element is expanded to the third diameter using a balloon member inserted within the lumen of the shunting element.

18. The method of any of examples 15-17 wherein adjusting the shunting element comprises compressing the shunting element to the third diameter, wherein the third diameter is smaller than the second diameter.

19. The method of example 18 wherein the shunting element is compressed to the third diameter using a balloon member positioned around an external surface of the shunting element.

20. A method of adjusting a shunting element in a septal wall between a left atrium and a right atrium in a patient, the method comprising:
advancing a sleeve around at least a portion of an outer circumference of the shunting element, the sleeve including one or more balloons in a first unexpanded configuration; and
expanding the one or more balloons to a second expanded configuration, wherein expanding the one or more balloons creates a radially inward force on the shunting element to decrease one or more dimensions of the shunting element.

21. The method of example 20, further comprising:
returning the one or more balloons to and/or toward the first unexpanded configuration; and
retracting the sleeve from around the at least portion of the outer circumference of the shunting element,
wherein the shunting element retains the one or more decreased dimensions following retraction of the sleeve.

22. The method of example 20 or 21 wherein the sleeve is configured to direct the one or more balloons to expand radially inward when expanding from the first unexpanded configuration to the second expanded configuration.

23. The method of example 22 wherein the sleeve is configured to prevent the one or more balloons from expanding radially outward when expanding from the first unexpanded configuration to the second expanded configuration.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the LA and RA, the LV and the right ventricle (RV), or the LA and the coronary sinus, it should be appreciated that similar embodiments could be utilized for shunts between other chambers of heart or for shunts in other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism coupled to the shunting element, wherein the anchoring mechanism is configured to secure the shunting element to a septal wall between the left atrium and the right atrium, and wherein the anchoring mechanism comprises one or more anchoring elements configured to penetrate into the septal wall.

2. The system of claim 1 wherein the one or more anchoring elements comprise one or more hooks or barbs.

3. The system of claim 1 wherein the anchoring mechanism further comprises a flange coupled to the shunting element, and the one or more anchoring elements are positioned on the flange.

4. The system of claim 1 wherein the one or more anchoring elements are positioned on an external surface of the shunting element.

5. The system of claim 1 wherein the one or more anchoring elements are positioned on an end portion of the shunting element.

6. The system of claim 1 wherein the shunting element comprises a stent having a plurality of struts.

7. The system of claim 1 wherein the anchoring mechanism further comprises one or more alignment features configured to limit a penetration depth of the one or more anchoring elements into the septal wall.

8. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism coupled to the shunting element, wherein the anchoring mechanism is configured to secure the shunting element to a septal wall between the left atrium and the right atrium, and wherein the anchoring mechanism comprises one or more inflatable flanges extending circumferentially around the shunting element.

9. The system of claim 8 wherein the one or more inflatable flanges comprise a first inflatable flange configured to be positioned against a left atrial side of the septal wall and a second inflatable flange configured to be positioned against a right atrial side of the septal wall.

10. The system of claim 8 wherein the one or more inflatable flanges are configured to receive a curable inflation medium.

11. A system for shunting blood between a left atrium and a right atrium of a patient, the system comprising:
a shunting element having a lumen extending therethrough, wherein the lumen is configured to fluidly couple the left atrium and the right atrium when the shunting element is implanted in the patient; and
an anchoring mechanism configured to secure the shunting element to a septal wall between the left atrium and the right atrium, the anchoring mechanism including:
a flange coupled to the shunting element and configured to be positioned against a right atrial side of the septal wall,
an anchoring element configured to be at least partially spaced apart from the septal wall, and
a tether connecting the shunting element to the anchoring element.

12. The system of claim 11 wherein the anchoring element comprises a stent positioned near or within a pulmonary vein.

13. The system of claim 11 wherein the anchoring element comprises an expandable structure.

14. The system of claim 13 wherein the expandable structure comprises a cage or a basket.

15. A method of implanting a shunting element in a septal wall between a left atrium and a right atrium in a patient, the method comprising:
positioning the shunting element around a balloon member while the shunting element is in a contracted configuration having a first diameter;
introducing the shunting element into or near an aperture in the septal wall;
expanding the shunting element to an expanded configuration having a second diameter by inflating the balloon member;
securing the shunting element to the septal wall using one or more anchoring elements; and
after securing the shunting element to the septal wall, adjusting the shunting element to a third diameter that is different than the second diameter.

16. The method of claim 15 wherein adjusting the shunting element comprises expanding the shunting element to the third diameter, wherein the third diameter is greater than the second diameter.

17. The method of claim 16 wherein the shunting element is expanded to the third diameter using a balloon member inserted within the lumen of the shunting element.

18. The method of claim 15 wherein adjusting the shunting element comprises compressing the shunting element to the third diameter, wherein the third diameter is smaller than the second diameter.

19. The method of claim 18 wherein the shunting element is compressed to the third diameter using a balloon member positioned around an external surface of the shunting element.

20. A method of adjusting a shunting element in a septal wall between a left atrium and a right atrium in a patient, the method comprising:
advancing a sleeve around at least a portion of an outer circumference of the shunting element, the sleeve including one or more balloons in a first unexpanded configuration; and
expanding the one or more balloons to a second expanded configuration, wherein expanding the one or more balloons creates a radially inward force on the shunting element to decrease one or more dimensions of the shunting element.

21. The method of claim 20, further comprising:
returning the one or more balloons to and/or toward the first unexpanded configuration; and
retracting the sleeve from around the at least portion of the outer circumference of the shunting element,
wherein the shunting element retains the one or more decreased dimensions following retraction of the sleeve.

22. The method of claim 20 wherein the sleeve is configured to direct the one or more balloons to expand radially inward when expanding from the first unexpanded configuration to the second expanded configuration.

23. The method of claim 22 wherein the sleeve is configured to prevent the one or more balloons from expanding radially outward when expanding from the first unexpanded configuration to the second expanded configuration.
